(19) United States
(12) Patent Application Publication
(10) Pub. No.: US 2022/0347044 A1
(43) Pub. Date: Nov. 3, 2022

(54) MASSAGE DEVICE

(71) Applicant: MTG CO., LTD., Nagoya-shi, Aichi (JP)

(72) Inventor: Tsuyoshi MATSUSHITA, Nagoya-shi (JP)

(73) Assignee: MTG CO., LTD., Nagoya-shi, Aichi (JP)

(21) Appl. No.: 16/651,562

(22) PCT Filed: Sep. 21, 2018
(86) PCT No.: PCT/JP2018/035073

§ 371 Date: Jun. 4, 2020

(30) Foreign Application Priority Data
Sep. 29, 2017 (JP) 2017-189963
Feb. 27, 2018 (JP) 2018-033901

(51) Int. Cl. A61H 15/00

(52) U.S. Cl. CPC A61H 15/0092; A61H 2015/0064

(57) ABSTRACT

A massager (1) includes a main body (2) that has a plate shape and applies a massage by sliding a peripheral edge (21) of the main body (2) on skin, and at least one rotating body (3). The rotating body (3) is disposed so as to protrude outward from the peripheral edge (21) of the main body (2). The rotation axis (31) of the rotating body (3) extends outward through the peripheral edge (21) of the main body (2).

### TECHNICAL FIELD

The present invention relates to a massager.

### BACKGROUND ART

As a massage method for beauty of the skin, there is known a method called “scraping”, which promotes blood flow and lymph flow by sliding the edge of a spatula or ceramic spoon, etc. to the skin, and increases the metabolism of the skin. In this type of massage method, a plate-shaped massager called a “scraping plate” may be used. The side surface of the scraping plate, that is, the peripheral edge of the scraping plate in plan view viewed from the thickness direction often has a complex shape in which a concave portion having a concavely curved contour in plan view, a convex portion having a convexly curved contour in plan view, and a linear portion having a linear contour in plan view are combined, so that various parts of the human body can be massaged.

As a massage method for beauty of the skin, a method for rolling while pressing a roller against the skin is also known. For example, Patent Literature 1 describes a massage tool having a plate-shaped main body and a plurality of rollers rotatably held on side surfaces of the main body.

### CITATION LIST

### Patent Literature

Patent Literature 1: Design Registration No. 1436400

### SUMMARY OF INVENTION

### Technical Problem

However, the rotation axis of the roller in the massage tool of Patent Literature 1 is disposed along the side surface of the scraping plate. Therefore, it is difficult to simultaneously perform the scraping and the massage with the roller by pressing the side surface of the main body and the roller against the skin. In other words, in order to simultaneously perform the scraping and the massage with the roller using the massage tool of Patent Literature 1, it is necessary to simultaneously press the roller and the peripheral edge of the roller on the side surface (that is, the peripheral edge) of the main body against the skin.

However, in the massage tool of Patent Literature 1, the plurality of rollers are disposed at equal intervals in a concavely curved portion on the side surface of the main body. In addition, a portion between the rollers on the side surface of the main body is located more inward than these rollers. Therefore, when the roller is pressed against the skin, the side surface of the main body hardly comes into contact with the skin, and in some cases, the side surface of the main body may not come into contact with the skin.

As described above, in the massage tool of Patent Literature 1, it is difficult to simultaneously press the roller and the side surface of the main body against the skin, and thus it is difficult to simultaneously perform the scraping and the massage with the roller.

Also, in the massage tool disclosed in Patent Literature 1, it may be difficult to press the roller against the skin when massaging a portion such as the face having relatively large changes in undulation.

An object of the invention is to provide a massager in which a rotating body can be pressed easily against a part with a comparatively large change of undulation while being able to perform scraping and massage with the rotating body simultaneously.

### Solution to Problem

Hereinafter, means for solving the above-described problems and the effects thereof will be described.

A massager to solve the above problem includes a main body that has a plate shape, and applies a massage by sliding a peripheral edge of the main body on the skin, and at least one rotating body that is rotatable and disposed to protrude outward from the peripheral edge of the main body. A rotation axis of the rotating body extends outward through the peripheral edge of the main body.

According to this configuration, the massager includes a rotating body which is disposed to protrude outward a side surface of the main body, that is, a peripheral edge of the main body in plan view viewed in the thickness direction. The rotation axis of the rotating body extends outward through the peripheral edge of the main body. By arranging the rotating body and the rotation axis in this manner, the peripheral edge of the main body and the rotating body can be pressed against the skin simultaneously. As a result, it is possible to simultaneously perform the massage by the rotating body and the scraping. In addition, by arranging the rotating body so as to protrude outward from the peripheral edge of the main body, the rotating body can be easily pressed against a portion such as a face where the undulation changes greatly.

### Advantageous Effects of Invention

According to the invention, a rotating body can be pressed easily against a part with a comparatively large change of undulation while being able to perform scraping and massage with the rotating body simultaneously.

### BRIEF DESCRIPTION OF DRAWINGS

FIG. 1 is a perspective view illustrating a massager of a first embodiment.

FIG. 2 is a plan view of the massager of the first embodiment viewed from a thickness direction.

FIG. 3 is a plan view illustrating a range of a concave portion and a convex portion in the massager of the first embodiment.

FIG. 4 is a plan view illustrating a longitudinal length and a lateral length of a main body in the massager of the first embodiment.

FIG. 5 is a side view of the massager according to the first embodiment viewed from a side where a rotating body is located.

FIG. 6 is a side view illustrating the angle of a rotation axis with respect to the thickness direction in the massager of the first embodiment.

FIG. 7 is a plan view of the massager according to the first embodiment as viewed such that a first vertex, a second vertex, and a rear end point of the main body are aligned in a straight line.

FIG. 8 is an explanatory view of a method for lifting up a cheek and an eye area using the massager of the first embodiment.

FIG. 9 is an explanatory view illustrating a part to be massaged by the method illustrated in FIG. 8.

FIG. 10 is an explanatory view of another method for lifting up the cheek and the eye area using the massager of the first embodiment.

FIG. 11 is an explanatory view illustrating a part to be massaged by the method illustrated in FIG. 10.

FIG. 12 is an explanatory view of a method for tightening the chin using the massager of the first embodiment.

FIG. 13 is an explanatory view illustrating a part to be massaged by the method illustrated in FIG. 12.

FIG. 14 is an explanatory view of a method for massaging the eye area with the rotating body using the massager of the first embodiment.

FIG. 15 is an explanatory view illustrating a part to be massaged by the method illustrated in FIG. 14.

FIG. 16 is an explanatory view of a method for performing massage so as to pick up the eye area by the rotating body by using the massager of the first embodiment.

FIG. 17 is an explanatory view illustrating a part to be massaged by the method illustrated in FIG. 16.

FIG. 18 is an explanatory view of a method for performing massage so as to pick up the outer corner of the eye with a rotating body using the massager of the first embodiment.

FIG. 19 is an explanatory view illustrating a part to be massaged by the method illustrated in FIG. 18.

FIG. 20 is an explanatory view illustrating an example of a state of contact between the peripheral edge of the main body and skin in a case of performing massage using the massager of the first embodiment.

FIG. 21 is an explanatory view illustrating an example of a state of contact between the peripheral edge of the main body and skin when massaging is performed using the massager in which another convex portion is connected to the convex portion from which the rotating body projects.

FIG. 22 is an explanatory view of a method for performing a massage for picking up the skin using a conventional cosmetic device.

FIG. 23 is an explanatory view of another method for performing massage for picking up skin using a conventional cosmetic device.

FIG. 24 is a plan view of a massager provided with rotating bodies at both ends of the concave portion in a second embodiment, as viewed from the thickness direction.

FIG. 25 is a plan view of a massager provided with a disk-shaped main body in a third embodiment, viewed from the thickness direction.

FIG. 26 is a plan view of the massager provided with a quadrangular-shaped main body in the third embodiment, viewed from the thickness direction.

FIG. 27 is a plan view of a massager including an arc-shaped main body in a fourth embodiment, as viewed from the thickness direction.

FIG. 28 is a plan view illustrating the longitudinal length and the lateral length of the main body in the massager of the fourth embodiment.

FIG. 29 is a plan view of the massager according to the fourth embodiment as viewed such that the first vertex, the second vertex, and the rear end point of the main body are aligned in a straight line.

FIG. 30 is a plan view of a massager provided with two rotating bodies at each of a first top portion and a second top portion in a fifth embodiment, viewed from the thickness direction.

FIG. 31 is a perspective view of a massager in which a first electrode, a second electrode, and a third electrode are provided in separate components in a sixth embodiment.

FIG. 32 is a plan view illustrating a range of a concave portion and a convex portion in the massager of the sixth embodiment.

### DESCRIPTION OF EMBODIMENTS

The main body of the massager embodying the invention has a plate shape. The massager is configured so that it can perform scraping by sliding its side surface against skin. In other words, the massager is configured to be able to perform scraping against the skin by sliding the peripheral edge of the massager in plan view viewed from the thickness direction thereof. Here, the plate-like shape includes a flat-like shape and a shape in which at least a part of the flat plate is curved to such an extent that the plate is recognized as a general feeling. For example, the surface of the portion of the main body surrounded by the peripheral edge in this example may be flat, may be slightly bulged in the thickness direction, or may be slightly depressed. In addition, the surface of the portion may be provided with irregularities. Further, the so-called “thickness direction” refers to a direction perpendicular to a plane where the projected area of the main body is maximized.

The shape of the main body in plan view viewed from the thickness direction, that is, in plan view in which the projected area of the main body is maximized, is not particularly limited, and may take various forms. For example, the shape of the main body in plan view may be a circle, an ellipse, a shape based on a circle such as an oval, or a triangle, a square, a rectangle, or may be a polygon such as a pentagon.

The main body may have a shape obtained by deforming the relatively simple shape described above to such an extent that the shape can be recognized as a general sense. Further, in addition to the above-described relatively simple shape, the main body has a relatively complex shape such as a heart or comma shape whose contour in plan view is a combination of straight and curved lines.

The massager includes at least one rotating body which is rotatable and disposed to protrude outward from the peripheral edge of the main body. The rotation axis of the rotating body extends outward through the peripheral edge of the main body. The main body includes at least one bulging portion bulging outward in plan view when viewed from the thickness direction thereof, and the rotating body is preferably disposed so as to protrude outward from the bulging portion. Here, the above-mentioned bulging portion includes a portion which can be recognized as bulging outward from the surroundings as a general sense, for example, a heart-shaped head, a comma-shaped head, a square corner, or the like.

By arranging the rotating body so as to protrude outward from the bulging portion, when the rotating body is pressed against the skin, the portion connected to the bulging portion together with the rotating body can be easily pressed against the skin. As a result, the usability of the massager can be further improved. Further, in this case, the rotating body can be easily pressed against the surface of the skin even at a portion depressed from the surroundings, such as an eye area and a nose line. As a result, massage by the rotating body can be performed evenly.

In addition, the main body may include two bulging portions disposed side by side with each other, and a depressed portion which is formed between these bulging portions and depressed inward from these bulging portions in plan view viewed from the thickness direction of the main body. In this case, a portion that protrudes outward from the periphery of the chin or the like is sandwiched in the depressed portion, that is, between the two bulging portions, and the entire depressed portion can be easily pressed against the surface of the corresponding portion. As a result, it is possible to perform scraping with uniformity in a portion that protrudes outward from the surroundings. Furthermore, in this case, while the portion that protrudes outward from the surroundings is sandwiched in the depressed portion, and the rotating body is pressed against the skin and the massager is reciprocated, the scraping and the massage by the rotating body can be performed simultaneously.

The peripheral edge of the main body includes at least one convex portion whose contour in plan view viewed from the thickness direction of the main body is convexly curved. At least one of a concave portion whose contour in plan view curves concavely and continues to the convex portion, and a linear portion whose contour in plan view is linear and continuous to the convex portion is provided by at least one. The rotation axis of the rotating body desirably extends outward through the convex portion. That is, it is preferable that the rotating body is disposed so as to protrude outward from the convex portion connected to either the concave portion or the linear portion.

If another convex portion is continuous with the convex portion on which the rotating body is disposed, and when the rotating body is pressed against the skin, the convex portion is disposed so as to move away from the skin as the distance from the rotating body increases. Therefore, in this case, when the rotating body is pressed against the skin, the length of the convex portion that can simultaneously contact the skin tends to be relatively short.

On the other hand, when the linear portion or the concave portion is continuous with the convex portion on which the rotating body is disposed, the linear portion or the concave portion is disposed at a position near the skin compared to a case where the other convex portion is continuous to the convex portion where the rotating body is disposed. Therefore, in this case, since the rotating body and the above-described concave portion or linear portion can be pressed against the skin simultaneously, the length of the contact portion between the peripheral edge of the main body and the skin can be made longer compared to a case where the rotating body and the convex portion are pressed against the skin simultaneously. As a result, scraping can be performed more efficiently.

Further, the peripheral edge of the main body includes a convex portion and a concave portion connected to the convex portion. The rotating body is desirably disposed so as to protrude outward from the convex portion. When the scraping and the massage with the rotating body are performed simultaneously, the surface of the portion where the scraping is, for example, often a convex curved surface such as a cheek or a chin. Therefore, by providing the concave portion adjacent to the convex portion on which the rotating body is disposed, when the rotating body is pressed against the skin, the concave portion is disposed along the convex curved surface of the skin. As a result, it is possible to press the concave portion along the curved shape of the skin while pressing the rotating body against the skin. Thereby, the length of the contact portion between the peripheral edge of the main body and the skin can be further lengthened, and the scraping can be performed more efficiently.

In addition, the peripheral edge of the main body includes, at least in a part, a concave portion or a linear portion, and a convex portion connected to both ends of the concave portion or the linear portion, respectively. It is preferable that the rotating body be disposed such that at least the part protrudes outward from a straight line contacting both of these convex portions. In this case, when the concave portion or the linear portion is pressed against the skin, the rotating body can protrude toward the skin. Therefore, the rotating body can be more easily pressed against the skin while the linear portion or the concave portion is pressed against the skin. Thereby, the usability of the massager can be further improved.

At least one rotating body is disposed so as to protrude outward from a side surface of the main body, that is, a peripheral edge of the main body in plan view viewed in the thickness direction. The rotating body may be disposed so as to entirely protrude from the peripheral edge of the main body, or may be disposed so that a part thereof protrudes from the peripheral edge of the main body. If at least a part of the rotating body is disposed so as to protrude from the peripheral edge of the main body, the peripheral edge of the main body and the rotating body can be pressed against the skin simultaneously.

The rotating body may be, for example, spherical or cylindrical. Here, the spherical shape includes a true sphere and a shape obtained by deforming the true sphere to the extent that it is recognized as a spherical shape as a general sense such as an elliptical sphere or a teardrop shape. In addition, the cylindrical shape includes a cylinder such as a circular tube and a square tube, and a shape obtained by deforming the cylinder to the extent that it is recognized as a spherical shape as a general sense such as a tapered shape, that is a shape having a smaller diameter near the tip or an inverse tapered shape, that is, a shape having a thicker diameter near the tip.

The outer diameter of the rotating body is not particularly limited. For example, in the case of a spherical rotating body, the diameter in a direction orthogonal to the rotation axis can be appropriately set within a range of 10 to 20 mm. In the case of a spherical rotating body, the diameter is preferably in a range of 15 to 20 mm. By setting the diameter of the spherical rotating body in a specific range, the rotating body can be more easily pressed against the surface of the skin even at a site that is depressed from the surroundings, such as an eye area or a nose line. From such a viewpoint, the diameter is more preferably in a range of 18 to 19 mm.

In the case of a cylindrical rotating body, for example, the diameter in a direction orthogonal to the rotation axis is appropriately set within a range of 10 to 20 mm, and the length in a direction along the rotation axis can be appropriately set within a range of 10 to 25 mm.

The surface of the rotating body may be configured by a curved surface, or may be configured by a large number of planes such as a triangular plane and a quadrangular plane. From the viewpoint of further improving the massage effect on the skin, it is preferable that the surface of the rotating body is configured by a large number of planes. Further, from the same viewpoint, it is preferable that a large number of protrusions are provided on the surface of the rotating body.

The rotating body preferably has a spherical shape. In this case, the rotating body can be easily pressed against the surface of the skin even at a portion depressed from the surroundings, such as an eye area and a nose line. As a result, massage by the rotating body can be performed evenly.

The rotation axis of the rotating body is a straight line that extends outward through the peripheral edge of the main body. For example, the rotation axis may extend through the center of gravity of the rotating body, or may not extend through the center of gravity of the rotating body. The rotation axis may extend in a direction orthogonal to the thickness direction of the main body, that is, in a direction parallel to a plane where the projected area of the main body is maximized, or in a direction inclined with respect to the thickness direction of the main body.

The angle between the thickness direction of the main body and the extending direction of the rotation axis can be appropriately set, for example, in a range of 60 to 120 degrees. Thereby, the peripheral edge of the main body and the rotating body can be pressed against the skin simultaneously, and furthermore, the rotating body can be rotated with the movement of the massager.

The angle between the thickness direction of the main body and the extending direction of the rotation axis is preferably in a range of 75 to 105 degrees, more preferably in a range of 80 to 100 degrees, still more preferably in a range of 85 to 95 degrees, and particularly preferably in a range of 88 to 92 degrees. As the angle between the thickness direction of the main body and the extending direction of the rotation axis approaches 90 degrees, the rotating body can be rotated more smoothly.

Further, the massager includes two rotating bodies disposed at an interval outside the main body, and the rotation axes of these two rotating bodies preferably extend as an interval therebetween is apart as going outward from the main body. In this case, by moving the massager back and forth on the skin while pressing the two rotating bodies against the skin, massage can be performed so that the skin is picked up by the rotating bodies. And, by using the massager, the user can select a desired massage from among a scraping, a massage in which the rotating body moves on the skin, a massage in which the scraping and the movement of the rotating body performed simultaneously, and a massage in which the skin is picked

Up.

In this case, the interval between the rotating bodies can be appropriately set according to the shape and the diameter of the rotating bodies. For example, when the rotating body has a spherical shape with a diameter of 10 to 20 mm, it is preferable to set the interval between the rotating bodies within a range of 7 mm or more and less than 10 mm. Thereby, the skin can be picked up moderately and strongly by the two rotating bodies, and the massage effect can be further improved. Further, it is preferable that the angle between the rotation axes is set within a range of, for example, 40 to 70 degrees. Also in this case, the skin can be picked up moderately and strongly by the two rotating bodies, and the massage effect can be further improved. From the viewpoint of further improving the massage effect, it is more preferable that the interval between the rotating bodies is 7.5 mm or more and 9 mm or less. Further, the angle between the rotation axes is more preferably 45 to 65 degrees, and even more preferably 50 to 60 degrees.

In this case, the massager may further include one or more rotating bodies in addition to the two rotating bodies for picking up the skin.

In the above case, the angle between the thickness direction of the main body and the extending direction of the rotation axis is preferably in a range of 60 to 120 degrees, more preferably in a range of 75 to 105 degrees, still more preferably in a range of 80 to 100 degrees, still more preferably in a range of 85 to 95 degrees, and particularly preferably in a range of 88 to 92 degrees.

In a case of performing a massage for picking up the skin, as described above, the massager is reciprocated on the skin while the two rotating bodies against the skin is pressed. In a case of performing the massage by this method, the magnitude of the force for picking up the skin changes according to the angle between the traveling direction of the massager and the extending direction of the rotation axis. Therefore, when a difference between the angle between the traveling direction on the forward path and the extending direction of the rotation axis and the angle on the backward path is large, the difference between the force for picking up the skin on the forward path and the force on the backward path becomes large. As a result, there is a possibility that the force for picking up the skin on one of the forward path and the backward path may be weaker than the force for picking up the skin on the other side.

By making the angle between the thickness direction of the main body and the extending direction of the rotation axis closer to 90 degrees, the difference between the angle between the traveling direction in the forward path and the extending direction of the rotation axis and the angle in the backward path is easily reduced. For this reason, a simple operation of reciprocating the massager can provide the same level of massage effect in the forward path and the backward path. As a result, the usability of the massager can be further improved.

In the above case, it is preferable that the two rotating bodies are disposed side by side along the contour of the main body in plan view viewed from the thickness direction. In a case of performing a massage for picking up the skin with two rotating bodies, as described above, while the two rotating bodies are pressed against the skin, the massager may be reciprocated in the direction orthogonal to the direction in which the two rotating bodies are aligned.

Conventionally, as a cosmetic device for picking up the skin with two rotating bodies, for example, a cosmetic device described in Japanese Patent Application Laid-Open No. 2015-186540 is known. The cosmetic device includes a rod-shaped handle and two rotating bodies provided at the tip of the handle. When two rotating bodies are pressed against the skin in this type of cosmetic device, the longitudinal direction of the handle is substantially orthogonal to the direction in which the two rotating bodies are disposed. Therefore, in a case of performing a massage for picking up the skin using this type of cosmetic device, it is necessary to move the entire arm and reciprocate the cosmetic device in the longitudinal direction of the handle.

On the other hand, when the two rotating bodies are disposed side by side along the contour of the main body as described above, when these rotating bodies are pressed against the skin, the thickness direction of the main body and the arranging direction of the two rotating bodies substantially form an orthogonal angle.

Therefore, for example, in a case of performing a massage for picking up the skin at a portion along the cheekbone or above the shoulder such as the eye area, the rotating body is pressed against the skin in a posture that the hand holding the massager is placed on the upper side and the elbow is placed on the lower side, and the massager may be reciprocated in the thickness direction such that the forearm and the wrist are swung right and left. Also, for example, in a case of performing a massage for picking up the skin at a portion below the shoulders such as the abdomen and arms, the rotating body is pressed against the skin in such a posture that the forearms are horizontal, and the massager may be reciprocated in the thickness direction by swinging the forearm and the wrist up and down.

As described above, according to the massager, a massage for picking up the skin can be performed by moving the forearm and the wrist in a swinging manner without moving the upper arm. As a result, it is possible to further reduce the load on the arm in a case of performing a massage for picking up the skin, and to improve the usability of the massager.

The massager includes two vertexes, an intersection between the rotation axes of the two rotating bodies, and a rear end point which is in a farthest distance from the intersection on the outer surface of the main body. One vertex of the two vertexes is a point at which the outer surface of one of the two rotating bodies intersects with the rotation axis of the rotating body, and the other of the two vertexes is a point where the outer surface of the other of the two rotating bodies intersects with the rotation axis of the rotating body. The vertex having a shorter distance from the rear end point of these two vertexes is referred to as a first vertex, and the vertex having a longer distance from the rear end point of the two vertexes is referred to as a second vertex. A direction parallel to a straight line connecting the second vertex and the rear end point is referred to as a longitudinal direction, and a direction orthogonal to the straight line is referred to as a lateral direction in plan view viewed from the thickness direction of the main body. In this case, it is preferable that the external dimension of the main body in the longitudinal direction be longer than the external dimension of the main body in the lateral direction.

Here, the external dimension in the longitudinal direction and the external dimension in the lateral direction are dimensions measured as follows. First, in plan view viewed from the thickness direction, that is, in plan view viewed so that the projected area of the main body is maximized, the main body is configured by a side extending in the longitudinal direction and a side extending in the lateral direction, and a rectangle abutting on the main body is defined. The longitudinal length of the rectangle is defined as the external dimension in the longitudinal direction, and the lateral length is defined as the external dimension in the lateral direction.

In a case of performing a massage for rolling the rotating body to the skin or a massage for picking up the skin, the user grasps the main body from the opposite side of the rotating body and presses the rotating body against the skin, so that the pressing force on the skin can be set moderately. At this time, by making the shape of the main body long in the longitudinal direction as described above, the distance from the hand to the rotating body can be reduced as compared with the case where the main body is long in the lateral direction. As a result, in a case of performing the above-described massage, the massager can be easily moved as intended while appropriately pressing the skin, and the usability of the massager can be further improved.

The main body includes two regions defined by the center line in plan view viewed from the thickness direction. The center line is a line connecting the centroids of the main body in a cross section perpendicular to a straight line connecting the second vertex and the rear end point. In other words, the center line is a line extending through the centroid of the main body in a cross section perpendicular to a straight line connecting the second vertex and the rear end point. In this case, the two rotating bodies may be disposed outside the same region of the two regions. Here, the centroid of the main body refers to the center of a region surrounded by the contour of the main body in the cross section.

In this case, in a case of performing a massage for rolling the rotating body to the skin or a massage for picking up the skin, the user grasps the main body from the opposite side of the rotating body and presses the rotating body against the skin, so that the pressing force on the skin can be set moderately. In addition, by disposing the rotating body at the specific position, the main body can be gripped in a natural posture with the wrist straight, and the rotating body can be pressed against the skin. As a result, the usability of the massager can be further improved.

The main body may have a tapered shape in which a width in plan view viewed from the thickness direction becomes narrower from the second vertex side toward the rear end point. In this case, the shape of the main body can be a shape that is more easily held by the user. As a result, the usability of the massager can be further improved.

The massager includes a first electrode disposed on the peripheral edge of the main body, a second electrode disposed on a surface of the rotating body, and a third electrode disposed on the main body at a position separated from the first electrode. It is preferable that a current path including the electrode in contact with the skin can be formed in a state where at least one of the first electrode and the second electrode and the third electrode are in contact with the skin.

In this case, the main body is grasped by hand so as to in contact with the third electrode, and at least one of the first electrode and the second electrode is pressed against the skin to perform a massage such as a scraping, so that a weak current can flow to the current path. In addition to the massage such as a scraping, the effect of the massage and the effect of the weak current act synergistically by applying the aforementioned weak current to the part being massaged, so that the cosmetic effect on the skin can be more improved.

The weak current flowing through the skin is not particularly limited. For example, the magnitude of the current can be set as appropriate from a range of 50 to 1000 μA. Further, the waveform of the current may be a direct current or an alternating current such as a sine wave, a rectangular wave, and a triangular wave. Further, the current may be a pulse current composed of one pulse or a plurality of pulse groups. By appropriately selecting the magnitude and waveform of the current from these ranges, for example, a weak current called a microcurrent for improving skin metabolism, an ion introducing current for causing a charged cosmetic ingredient to penetrate from the skin surface to the inside of the skin, or an ion deriving current for causing a charged dirt component such as dirt on the skin to float from the inside of the skin to the skin surface can flow through the skin.

### EMBODIMENTS

### First Embodiment

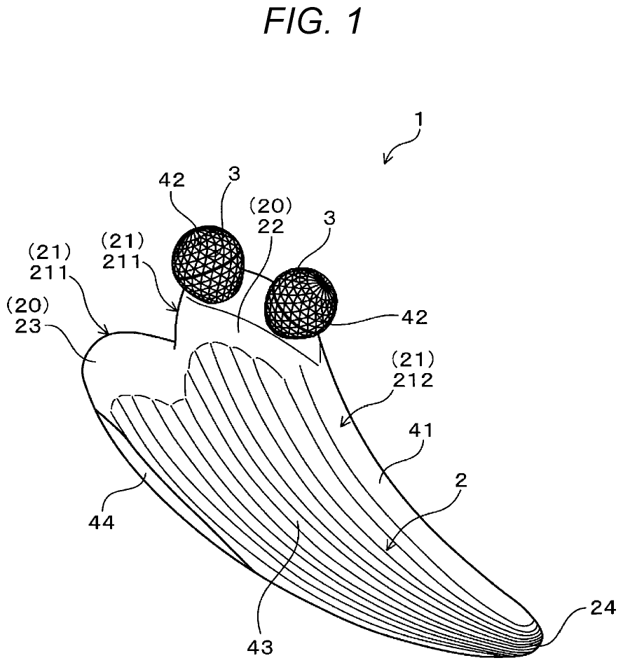

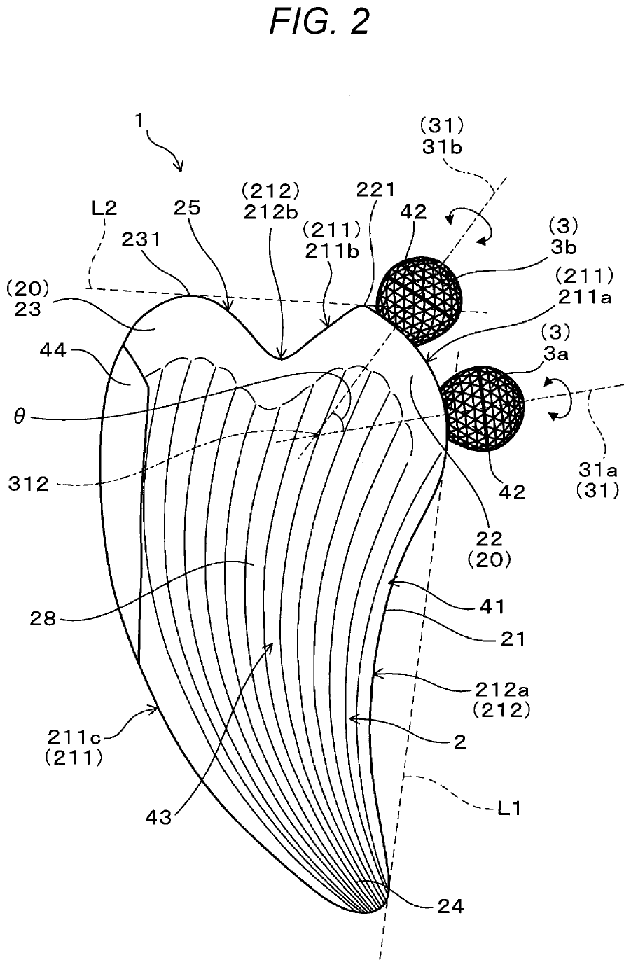

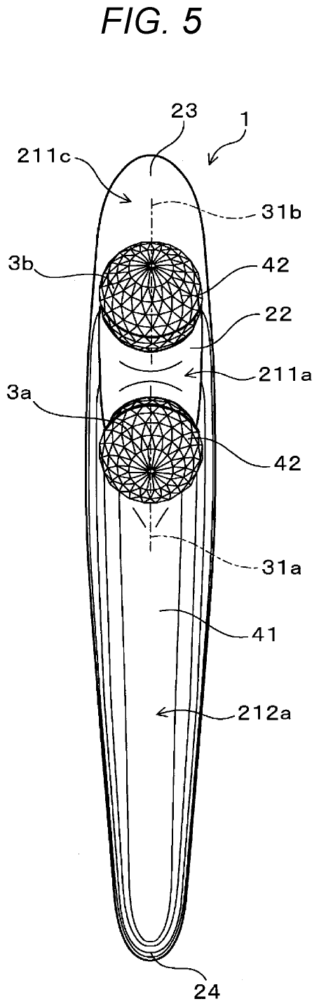

A massager 1 according to a first embodiment will be described with reference to FIGS. 1 to 23. As illustrated in FIGS. 1 to 5, the massager 1 includes a main body 2 that has a plate-like shape and applies a massage by sliding a peripheral edge 21 of the main body 2 on the skin, and at least one rotating body 3 disposed so as to protrude from an outer side from the peripheral edge 21 of the main body 2. The massager 1 of the first embodiment includes two rotating bodies 3. As illustrated in FIGS. 2 and 5, a rotation axis 31 of the rotating body 3 extends outward through the peripheral edge 21 of the main body 2.

As illustrated in FIGS. 1 and 2, the main body 2 of the massager 1 has a heart shape in plan view viewed from the thickness direction thereof, that is, in plan view when the projected area is maximized (see FIG. 2). More specifically, the main body 2 includes two top portions 22 and 23 disposed side by side with each other, and a tail portion 24 extending from the top portions 22 and 23. Each of the top portions 22 and 23 bulges outward so as to have a substantially hemispherical shape in plan view. The width of the tail portion 24 becomes narrower as it approaches the tip.

Hereinafter, the direction in which the two top portions 22 and 23 are disposed may be referred to as a width direction. In addition, the direction orthogonal to both the width direction and the thickness direction may be referred to as a vertical direction, and a side near the top portions 22 and 23 in the vertical direction may be referred to as an upper side, and a side near the tail portion 24 may be referred to as a lower side. These descriptions regarding the direction of the massager 1 are for convenience, and have nothing to do with the direction in which the massager 1 is used.

Each of the top portions 22 and 23 is provided as a bulging portion 20 bulging outward in plan view viewed from the thickness direction of the main body 2. The rotation axis 31 of the rotating body 3 extends outward through one top portion 22 of the top portions 22 and 23. Further, between the two top portions 22 and 23, there is formed a depressed portion 25 which is depressed inward from the top portions 22 and 23 in plan view viewed from the thickness direction of the main body 2. In the following, for convenience, one of the two top portions 22 and 23 where the rotating body 3 is provided is referred to as a first top portion 22 and the other top is referred to as a second top portion 23. Further, the first top portion 22 side in the width direction may be referred to as a right side, and the second top portion 23 side may be referred to as a left side. The description about these left-right directions is for convenience, and has nothing to do with the orientation when using the massager 1.

As illustrated in FIG. 2, the tail portion 24 of the main body 2 is curved toward the first top portion 22 in the width direction from a side of the top portions 22 and 23 toward the tip of the tail portion 24.

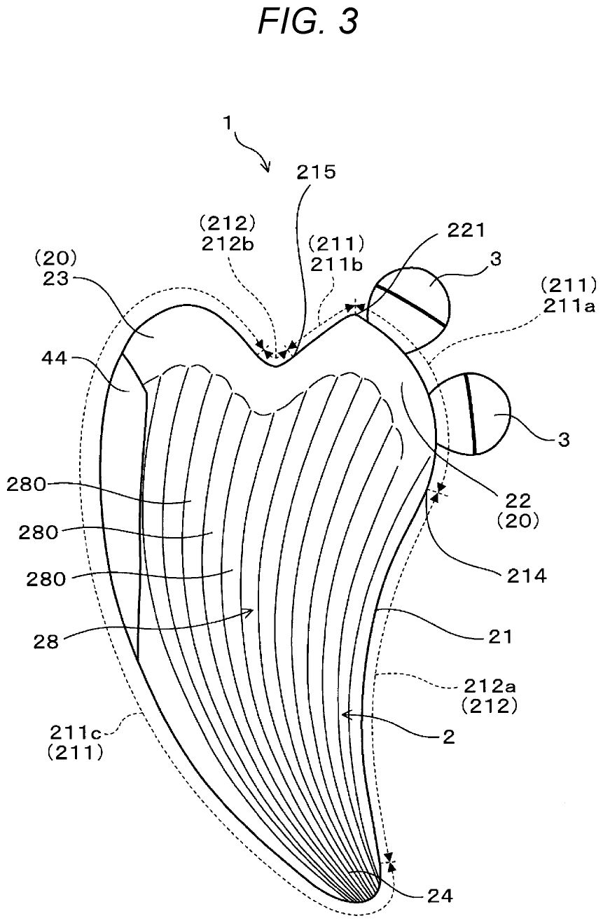

Further, as illustrated in FIGS. 2 and 3, the outer surface of the main body 2 includes a peripheral edge 21 defining a contour of the main body 2 in plan view viewed from the thickness direction, and two main surface portions 28 surrounded by the peripheral edge 21. In the first embodiment, the peripheral edge 21 of the main body 2 includes three convex portions 211 and two concave portions 212. As illustrated in FIG. 3, the three convex portions 211 are portions whose contours are convexly curved in plan view viewed from the thickness direction of the main body 2, and include a first convex portion 211a, a second convex portion 211b, and a third convex portion 211c. The two concave portions 212 are portions where the contour in plan view curves in a concave shape and continues to the convex portion 211, and is configured by a first concave portion 212a and a second concave portion 212b. In addition, the rotation axis 31 of the rotating body 3 extends outward through the convex portion 211. In FIG. 3, the rotating body 3 is simplified for convenience.

The configuration of the peripheral edge 21 of the main body 2 will be described more specifically with reference to FIG. 3. The peripheral edge of the first top portion 22 includes the first convex portion 211a forming an outer portion in the width direction (that is, a right portion) and the second convex portion 211b forming an inner portion in the width direction (that is, a left portion). The first convex portion 211a is connected to the second convex portion 211b at the upper end 221 of the first top portion 22. In addition, the two rotating bodies 3 are disposed on the first convex portion 211a so as to protrude outward from the first convex portion 211a. The second convex portion 211b has a larger radius of curvature than the first convex portion 211a.

Between the peripheral edge of the first top portion 22 and the peripheral edge of the tail portion 24, that is, in a portion on the first top portion 22 side in the width direction in the peripheral edge 21 of the main body 2, the first concave portion 212a is disposed. The first concave portion 212a connects the first convex portion 211a and a third convex portion 211c described later. As described above, the peripheral edge 21 of the main body 2 includes the first convex portion 211a and the first concave portion 212a connected to the first convex portion 211a. The rotation axis 31 of the rotating body 3 extends outward through the first convex portion 211a.

The second concave portion 212b is disposed between the first top portion 22 and the second top portion 23. The second concave portion 212b connects the first convex portion 211a and the second convex portion 211b.

The remaining portion of the peripheral edge 21 of the main body 2, that is, a portion extending from the peripheral edge of the second top portion 23 to the tail portion 24 via the portion of the peripheral edge 21 of the main body 2 on the side of the second top portion 23 in the width direction includes the third convex portion 211c.

In other words, the first convex portion 211a is disposed on the peripheral edge 21 of the main body 2 to the right of the upper end 221 of the first top portion 22. The contour of the first convex portion 211a in plan view viewed from the thickness direction of the main body 2 swells rightward as it goes downward from the upper end 221 of the first top portion 22, and then has a convex shape that retreats leftward.

The first concave portion 212a is continuous with a lower end 214 of the first convex portion 211a. The first concave portion 212a is disposed over a range from the first top portion 22 to the tail portion 24 in the peripheral edge 21 of the main body 2. The contour of the first concave portion 212a in plan view has a concave shape that retreats leftward downward from the lower end 214 of the first convex portion 211a and then expands rightward.

The second convex portion 211b is continuous with the upper end of the first convex portion 211a. Specifically, the second convex portion 211b is disposed on the left side of the upper end 221 of the first top portion 22 in the peripheral edge 21 of the main body 2. The contour of the second convex portion 211b in plan view extends from the upper end 221 of the first top portion 22 toward a position between the first top portion 22 and the second top portion 23. The contour of the second convex portion 211b in plan view has a convex shape in which an intermediate portion between the upper end 221 of the first top portion 22 and the left end 215 of the second convex portion 211b slightly bulges outward.

The second concave portion 212b is continuous with a left end 215 of the second convex portion 211b. The second concave portion 212b is disposed between the first top portion 22 and the second top portion 23. The contour of the second concave portion 212b in plan view has a concave shape that is depressed downward from the left end 215 of the second convex portion 211b to the left, and then protrudes upward.

The third convex portion 211c forms the remaining portion of the peripheral edge 21 of the main body 2. That is, the third convex portion 211c forms a portion of the peripheral edge 21 from the portion corresponding to the second top portion 23 to the tail portion 24 via the left portion of the peripheral edge 21. The contour of the third convex portion 211c in plan view is convexly curved over the entire range.

Further, the main body 2 includes a main surface portion 28 inside the peripheral edge 21 in plan view viewed from the thickness direction. The main surface portion 28 includes a plurality of raised portions 280 extending from the tail portion 24 toward the top portions 22 and 23. The plurality of raised portions 280 have a curved shape curved along the contour of the main body 2 in plan view viewed from the thickness direction. Further, although not illustrated in the drawing, each raised portion 280 has an arc-shaped contour in a cross section in the width direction.

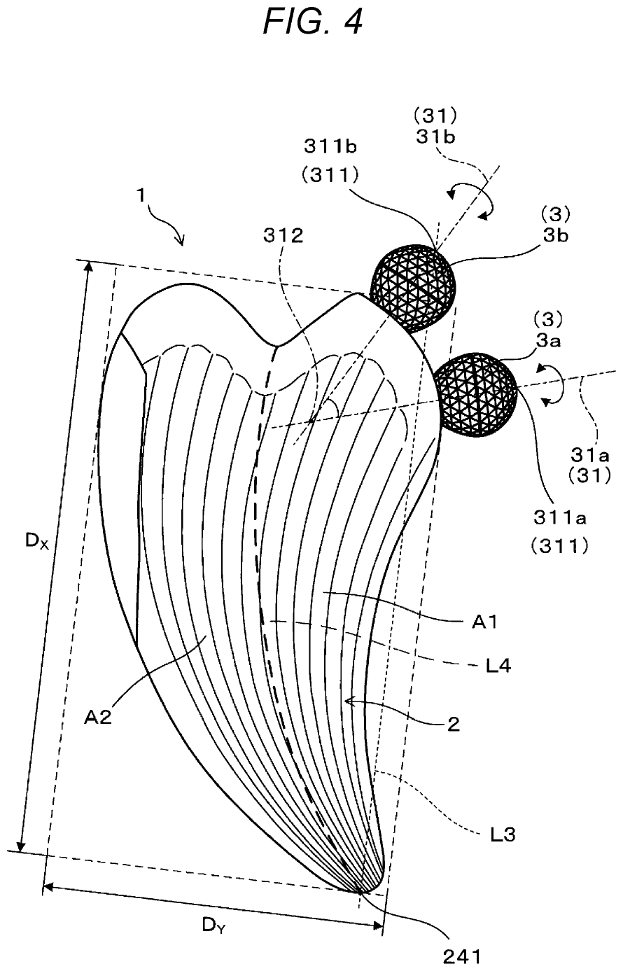

The massager 1 of the first embodiment includes two rotating bodies 3 (3a, 3b) disposed outside the first top portion 22 of the main body 2 with a space therebetween. The rotation axis 31 (31a, 31b) of each rotating body 3 (3a, 3b) extends through the center of gravity of the rotating body 3. In addition, the rotation axes 31 of these two rotating bodies 3 extend so that the interval between them increases as going outward from the main body 2, and as illustrated in FIGS. 2 and 4, the rotation axes 31 intersect with each other at an intersection 312. In the following, for convenience, the rotating body closer to the first concave portion 212a of the two rotating bodies 3 is referred to as a first rotating body 3a, and the rotating body closer to the second concave portion 212b is referred to as a second rotating body 3b.

The distance between the rotating bodies 3 in the first embodiment, that is, the minimum value of the distance between the surface of the first rotating body 3a and the surface of the second rotating body 3b can be appropriately set, for example, from a range of 7 to 10 mm. Further, an angle θ between the rotation axes 31 of the rotating body 3 can be appropriately set within a range of 40 to 70 degrees. In the massager 1 of the first embodiment, the interval between the rotating bodies 3 is 8.3 mm, and the angle θ between the rotation axes 31 of the rotating bodies 3 is 53 degrees.

As illustrated in FIG. 2, the first rotating body 3a is disposed to protrude outward from a straight line L1 that is in contact with both the first convex portion 211a and the portion below the third convex portion 211c (that is, the peripheral edge of the tail portion 24). Further, the second rotating body 3b is disposed to protrude outward of a straight line L2 that is in contact with both the second convex portion 211b and the portion above the third convex portion 211c (that is, the peripheral edge of the second top portion 23).

As described above, the peripheral edge 21 of the main body 2 in the massager 1 includes, at least in part, two convex portions 211 and the concave portion 212 disposed between the convex portions 211. Each of the rotating bodies 3 is disposed so that at least a part thereof protrudes outward from straight lines L1 and L2 in contact with both of the two convex portions 211.

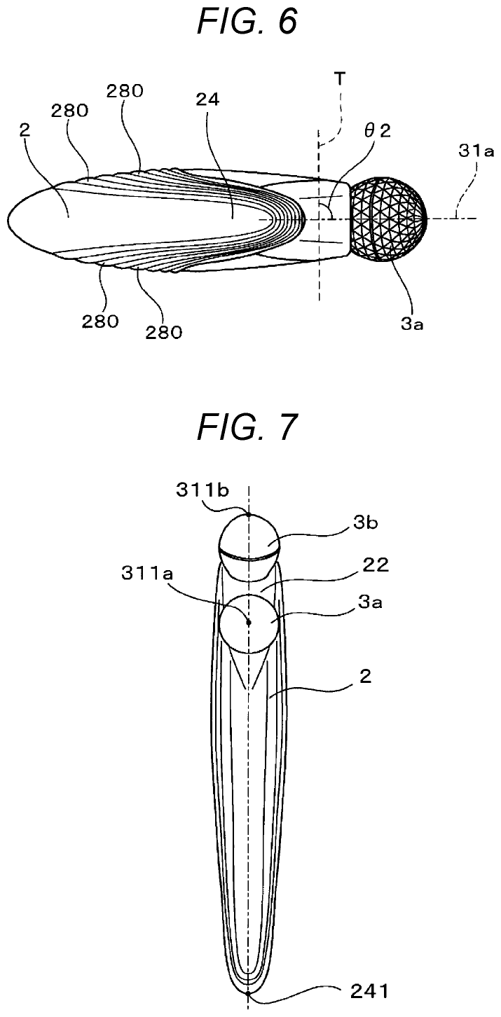

In addition, as illustrated in FIGS. 5 and 6, the rotation axis 31 of each rotating body 3 extends in a direction orthogonal to the thickness direction of the main body 2. That is, as illustrated in FIG. 6, an angle θ2 between the extending direction of the rotation axis 31 in each rotating body 3 and a straight line T parallel to the thickness direction of the main body 2 is 90 degrees.

Each of the rotating bodies 3 has a spherical shape as illustrated in FIGS. 1 to 5. More specifically, as illustrated in FIGS. 1 and 2, the rotating body 3 has a shape such that a sharp part in a teardrop shape is cut off. In other words, the rotating body 3 has a hemispherical half on the distal end side, and the outer diameter is gradually reduced from the hemispherical portion toward the main body 2 as compared with the case where the rotating body 3 is completely spherical shape. The diameter of the rotating body 3, that is, a maximum value of the outer diameter of the rotating body 3 in a direction orthogonal to the rotation axis 31 can be appropriately set, for example, in a range of 10 to 20 mm. The surface of the rotating body 3 is configured by a large number of triangular planes. On the surface of each rotating body 3, a second electrode 42 described later is provided.

The massager 1 of the first embodiment includes a first electrode 41 disposed on the peripheral edge 21 of the main body 2, the second electrode 42 disposed on the surface of the rotating body 3, and a third electrode 43 disposed at a position separated from the first electrode 41 in the main body 2. Then, in a state where at least one of the first electrode 41 and the second electrode 42 and the third electrode 43 are in contact with the skin, the massager 1 can form a current path including the electrode in contact with the skin.

More specifically, the first electrode 41 is provided over the entirety of the first concave portion 212a in the peripheral edge 21 of the main body 2, that is, the portion connecting the first top portion 22 provided with the rotating body 3 and the tail portion 24. Further, the third electrode 43 is provided in a portion inside the peripheral edge 21 of the main body 2 in plan view viewed from the thickness direction. The first electrode 41, the second electrode 42, and the third electrode 43 are connected to each other via a control circuit (not illustrated) built in the main body 2. In addition, the control circuit is connected to a solar cell module 44 disposed between the second top portion 23 and the tail portion 24 in the peripheral edge 21 of the main body 2.

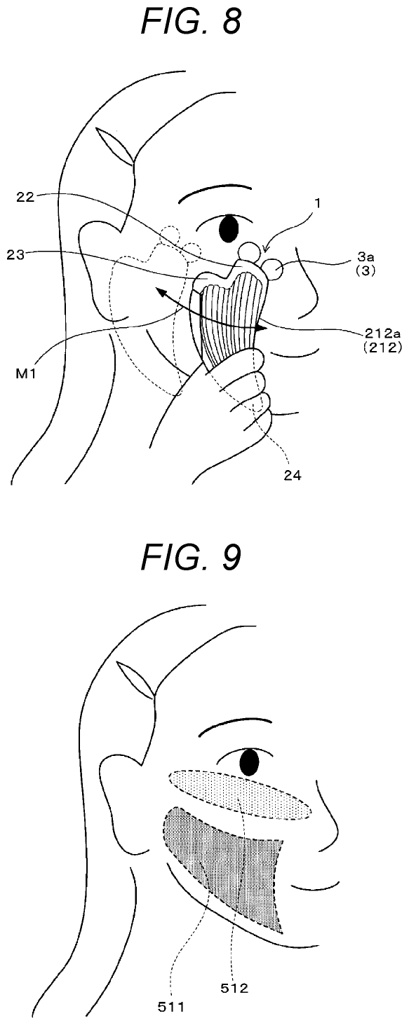

The massager 1 of the first embodiment can be used in various modes. For example, in a case of performing lift-up of the cheek and the eye area, as illustrated in FIG. 8, the first concave portion 212a of the main body 2 is pressed against the cheek along a nasolabial fold and the first rotating body 3a is pressed against the eye area. Then, the massager 1 is reciprocated right and left along the cheek (see arrow M1 in FIG. 8), so that the first concave portion 212a slides along the bulge of the cheek and the first rotating body 3a can be rolled in the eye area. Accordingly, as illustrated in FIG. 9, a cheek 511 having relatively thick skin can be given the scraping to promote blood flow. Further, at the same time as the scraping, the rotating body 3 can be rolled to the eye area 512 having relatively thin skin, and a soft stimulus such as massage by an esthetician can be applied to loosen the eye area. As a result, the cheek can be lifted up.

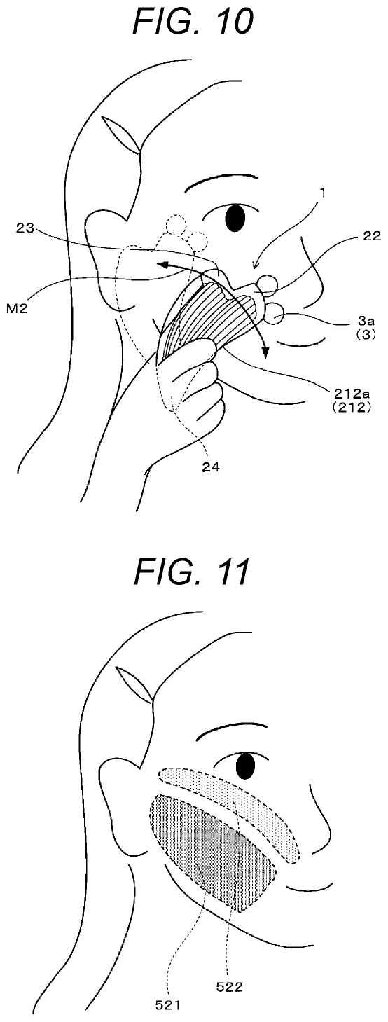

As another method for lifting up the cheek and eye area, for example, as illustrated in FIG. 10, the top portions 22 and 23 are directed toward the mouth, and the tail portion 24 is directed obliquely downward from the top portions 22 and 23, so that the first concave portion 212a is pressed against the cheek and the first rotating body 3a is pressed against the eye area. Then, by moving the massager 1 reciprocally (see arrow M2 in FIG. 10) so that the first rotating body 3a rotates around the tail portion 24, the first concave portion 212a slides along the bulge of the cheek, and at the same time, the first rotating body 3a can be rolled along the cheekbone. As a result, as illustrated in FIG. 11, a cheek 521 having relatively thick skin can be scraped to promote blood flow. Furthermore, simultaneously with the scraping, the rotating body 3 can be rolled to a portion 522 along the cheekbone with relatively thin skin, and a soft stimulus such as massage by an esthetician can be applied to loosen the portion. As a result, the cheek can be lifted up.

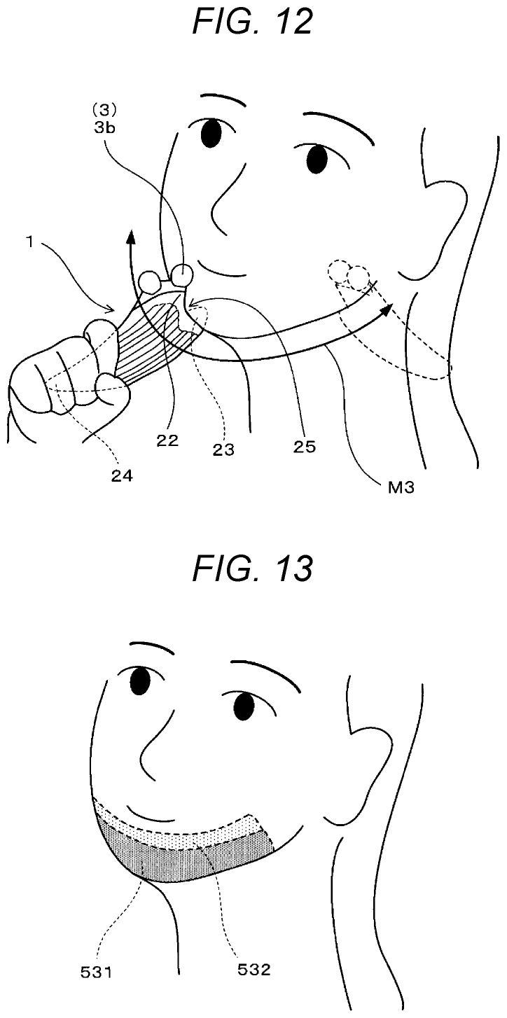

When tightening the chin, for example, as illustrated in FIG. 12, the main body 2 and the second rotating body 3b are pressed against the face by sandwiching the chin between the first top portion 22 and the second top portion 23 while pointing the second rotating body 3b toward the mouth. Then, by reciprocating the massager 1 along the chin (see arrow M3 in FIG. 12), the portion extending from the upper end 221 of the first top portion 22 to the upper end 231 of the second top portion 23, in the depressed portion 25 between the first top portion 22 and the second top portion 23, that is, in the peripheral edge 21 of the main body 2, can be slid along the chin, and at the same time, the second rotating body 3b can be rolled along the mouth. As a result, as illustrated in FIG. 13, a portion 531 along the chin having relatively thick skin can be scraped to promote blood flow. Further, at the same time as the scraping, the rotating body 3 can be rolled to the mouth 532 having relatively thin skin, and a soft stimulus such as massage by an esthetician can be applied to loosen the relevant portion. As a result, the chin can be tightened.

As described above, in addition to the method for simultaneously pressing the peripheral edge 21 of the main body 2 and the rotating body 3 against the skin to perform massage, the massager 1 can implement a method for performing the scraping along or a method for performing massage alone by the rotating body 3 may be implemented. For example, in the case of performing the scraping alone, although not illustrated in the drawing, the second top portion 23 and the tail portion 24 in the main body 2 are pressed against the skin to reciprocate the massager 1, so that the main body 2 gives an appropriate stimulus to the pressing portion to promote blood flow.

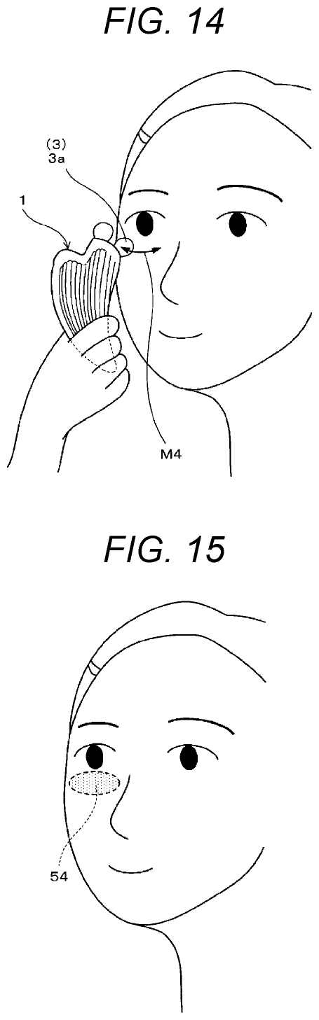

In a case of performing massage by the rotating body 3 alone, for example, as illustrated in FIG. 14, the first rotating body 3a is pressed against the eye area, and the massager 1 reciprocates left and right (see arrow M4 in FIG. 14), so that a soft stimulus such as massage by an esthetician can be applied to the portion 54 along the relatively thin eye area of the skin as illustrated in FIG. 15 to loosen the portion. Further, the same operation and effect can be obtained by rolling the second rotating body 3b on the skin instead of the method for rolling the first rotating body 3a on the skin as illustrated in FIG. 14.

Although not illustrated in the drawing, the massage by the rotating body 3 is not limited to the above-described eye area, and may be performed on a relatively thin skin area such as a part along the cheekbone or the mouth.

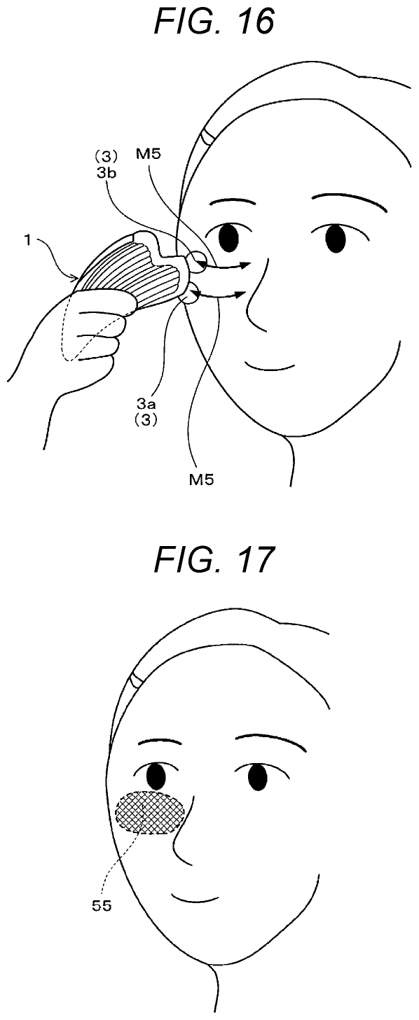

The massager 1 of the first embodiment can perform a massage for picking up the skin between the two rotating bodies 3 by pressing and reciprocating both the first rotating body 3a and the second rotating body 3b against the skin. For example, in a case of performing a massage for picking up the skin around the eye area, as illustrated in FIG. 16, the first rotating body 3a and the second rotating body 3b are disposed in the vertical direction to press these rotating bodies 3 against the eye area. Then, in this state, by reciprocating the massager 1 left and right along the eye area (see arrow M5 in FIG. 16), a skin 55 at the eye area (see FIG. 17) can be picked up to promote blood flow.

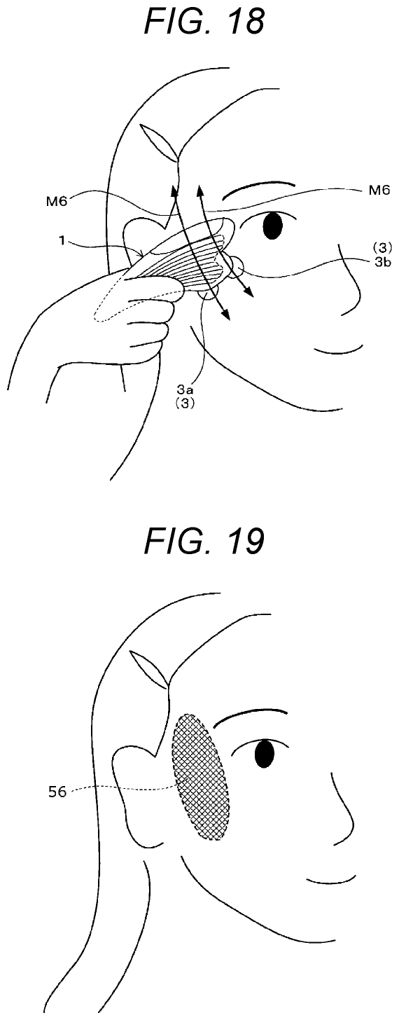

In addition, in a case of performing a massage for picking up the skin of the outer corner of the eye, as illustrated in FIG. 18, the first rotating body 3a and the second rotating body 3b are disposed in the left-right direction to press these rotating bodies 3 against the eye area. Then, in this state, by reciprocating the massager 1 along the line from the outer corner of the eye to the temple (see arrow M6 in FIG. 18), the skin 56 at the outer corner of the eye (see FIG. 19) can be picked up to promote blood flow.

Although not illustrated in the drawing, the massage for picking up the skin by the two rotating bodies 3 is not limited to the above-described eye area and the corner of the eye, but may be performed on a relatively thick portion of the skin such as a cheek, or may be performed on a relatively thin part of the skin such as a portion along the cheekbones and a mouth.

As described above, the massager 1 is configured to be able to perform various massages by appropriately changing the way of pressing the skin.

When applying a weak current to the skin by the massager 1, the main body 2 is gripped such that the third electrode 43 it touched by the hand, and at least one of the rotating body 3 and the first concave portion 212a comes into contact with the skin. As a result, a current path including at least one of the first electrode 41 and the second electrode 42 and the third electrode 43 is formed. When the solar cell module 44 is irradiated with light, power is supplied to the control circuit, and a weak current can flow through the current path. As a result, a weak current can flow through the skin.

Next, the operational effects of the massager 1 of the first embodiment will be described. As illustrated in FIG. 2, the massager 1 includes the rotating body 3 disposed so as to protrude outward from the peripheral edge 21 of the main body 2. The rotation axis 31 of the rotating body 3 extends outward through the peripheral edge 21 of the main body 2. By arranging the rotating body 3 and the rotation axis 31 in this manner, the peripheral edge 21 of the main body 2 and the rotating body 3 can be pressed against the skin simultaneously. As a result, it is possible to simultaneously perform the massage by the rotating body and the scraping. In addition, by arranging the rotating body 3 so as to protrude outward from the peripheral edge 21 of the main body 2, the rotating body can be easily pressed against a portion such as a face where the undulation changes greatly.

Further, as illustrated in FIG. 2, the main body 2 includes the tail portion 24, the first top portion 22 and the second top portion 23 as the bulging portions 20 bulging outward in plan view viewed from the thickness direction of the main body 2. The rotation axis 31 of the rotating body 3 extends outward through the first top portion 22 as the bulging portion 20. Therefore, for example, when the first rotating body 3a is pressed against the skin as illustrated in FIGS. 8 and 10, the first rotating body 3a and the first concave portion 212a connected to the first top portion 22 can be easily pressed on the skin. Also, for example, as illustrated in FIG. 12, when the second rotating body 3b is pressed against the skin, the first top portion 22, and the second concave portion 212b and the second top portion 23 connected to the first top portion 22 can be easily pressed against the skin. Therefore, the usability of the massager 1 can be further improved. Furthermore, since the rotating body 3 is disposed so as to protrude outward from the bulging portion 20, the rotating body 3 can be easily pressed against the surface of the skin even in a portion depressed from the surroundings, for example, around the eye area or a nose line. As a result, massage by the rotating body 3 can be performed evenly.

Further, as illustrated in FIG. 2, the main body 2 includes, as the bulging portion 20, the first top portion 22 and the second top portion 23 disposed side by side with each other. Further, the main body 2 includes the depressed portion 25 formed between the first top portion 22 and the second top portion 23. The depressed portion 25 is depressed more inward than the top portions 22 and 23 in plan view viewed from the thickness direction of the main body 2. Therefore, as illustrated in FIG. 12, for example, a portion that protrudes outward from the periphery of the chin or the like can be sandwiched in the depressed portion 25, and the entire depressed portion 25 can be easily pressed against the surface of the corresponding portion. As a result, it is possible to perform scraping with uniformity in a portion that protrudes outward from the surroundings. Furthermore, in this case, while the portion that protrudes outward from the surroundings is sandwiched in the depressed portion 25, and the rotating body 3 is pressed against the skin and the massager 1 is reciprocated, the scraping and the massage by the rotating body 3 can be performed simultaneously.

As illustrated in FIG. 2, the peripheral edge 21 of the main body 2 includes the convex portion 211 and the concave portion 212 connected to the convex portion 211. The rotating body 3 is disposed so as to protrude outward from the convex portion 211. Therefore, as illustrated in FIGS. 8 and 10, for example, it is possible to press the concave portion 212 along the curved shape of the skin while pressing the rotating body 3 against the skin.

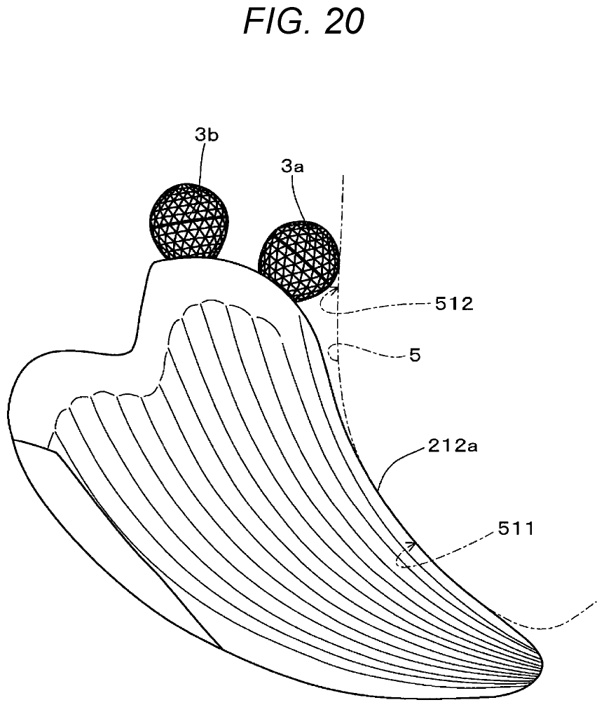

For example, in a case of performing lift-up of the cheek (see FIG. 8), as illustrated in FIG. 20, the first rotating body 3a is brought into contact with the eye area 512 and the first concave portion 212a is brought into contact with the cheek 511, so that massage is performed. At this time, since the first concave portion 212a is curved along the convex curved surface of the cheek 511, the first concave portion 212a can be pressed along the cheek 511.

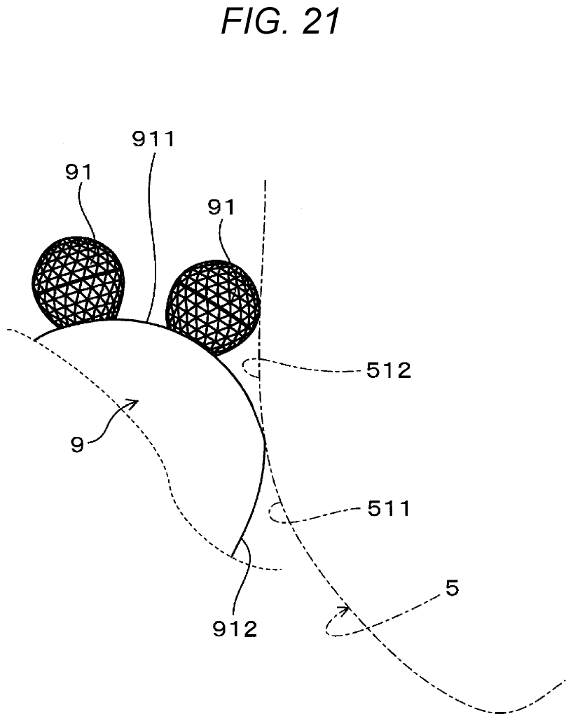

On the other hand, FIG. 21 illustrates a massager 9 which includes a convex portion 911, two rotating bodies 91 respectively protruding from the convex portion 911, and a convex portion 912 connected to the convex portion 911. The convex portion 912 is curved so as to be away from the convex curved surface of the cheek 511. Therefore, for example, when the scraping and the massage with the rotating body 91 are performed simultaneously using the massager 9, the distance from the skin to the convex portion 912 is easily made small compared with the massager 1 of the first embodiment (see FIG. 20). In FIGS. 20 and 21, a skin 5 including the cheek 511 and the eye area 512 is indicated by a dashed line.

As described above, in the massager 1, since the convex portion 211 and the concave portion 212 are disposed as described above, the length of the contact portion between the peripheral edge 21 of the main body 2 and the skin can be further increased. As a result, the scraping can be performed more efficiently.

Further, as illustrated in FIG. 2, the peripheral edge 21 of the main body 2 includes, at least in part, two convex portions 211 and the concave portion 212 disposed between the convex portions 211. The rotating body 3 is disposed so that at least a part thereof protrudes outward from straight line in contact with both of the two convex portions 211. Therefore, as illustrated in FIGS. 8 and 10, for example, when the concave portion 212 or the convex portion 211 is pressed against the skin, the rotating body 3 can protruded toward the skin. Therefore, the rotating body 3 can be more easily pressed against the skin while the linear portion or the concave portion 212 is pressed against the skin. Thereby, the usability of the massager 1 can be further improved.

The rotating body 3 has a spherical shape as illustrated in FIG. 2. Therefore, as illustrated in FIGS. 8, 10, and 14, for example, the rotating body 3 can be easily pressed against the surface of the skin even at a portion that is depressed from the surroundings, such as the eye area and the nose line. As a result, massage by the rotating body 3 can be performed evenly.

As illustrated in FIG. 5, the angle between the thickness direction of the main body 2 and the extending direction of the rotation axis 31 is in a range of 60 to 120 degrees. Therefore, for example, as illustrated in FIGS. 8 to 12, the peripheral edge 21 of the main body 2 and the rotating body 3 can be pressed against the skin simultaneously, and furthermore, the rotating body 3 can be rotated with the movement of the massager 1.

Further, the two rotating bodies 3 are disposed side by side along the contour of the main body 2 in plan view viewed from the thickness direction. In other words, the massager 1 of the first embodiment includes two vertexes 311 (311a, 311b), the intersection 312, and a rear end point 241 as illustrated in FIG. 4. One vertex (311a) of the two vertexes 311 is a point at which the outer surface of one rotating body (3a) of the two rotating bodies 3 intersects with the rotation axis (31a) of the rotating body (3a). The other vertex (311b) of the two vertexes 311 is a point at which the outer surface of the other rotating body (3b) of the two rotating bodies 3 intersects with the rotation axis (31b) of the rotating body (3b). The intersection 312 is an intersection between the rotation axes 31 (31a, 31b) of the two rotating bodies 3. The rear end point 241 is a point on the outer surface of the main body 2 where the distance from the intersection 312 is the largest.

Then, in the two vertexes 311 (311a, 311b), the vertex whose distance from the rear end point 241 is short (that is, the vertex of the first rotating body 3a) is referred to as a first vertex 311a, and the vertex whose distance from the rear end point 241 is long (that is, the vertex of the second rotating body 3b) is referred to as a second vertex 311b. As illustrated in FIG. 7, the first vertex 311a, the second vertex 311b, and the rear end point 241 are aligned in a straight line, and the first vertex 311a is located between the second vertex 311b and the rear end point 241 when viewed the massager 1 such that the distance from the second vertex 311b to the rear end point 241 is maximized. Although the surface shape of the rotating body 3 is simplified in FIG. 7 for convenience, the surface of the rotating body 3 is actually configured by a large number of triangular planes as illustrated in FIGS. 1 to 5.

The massager 1 of the first embodiment, for example, as illustrated in FIGS. 16 and 18, can moderately and strongly pick up the skin by the two rotating bodies 3 by pressing the two rotating bodies 3 against the skin and reciprocating the massager 1 in the thickness direction of the main body 2. Further, the reciprocating movement described above can be easily realized, for example, by moving the massager 1 so as to swing the forearm or the wrist without moving the upper arm.

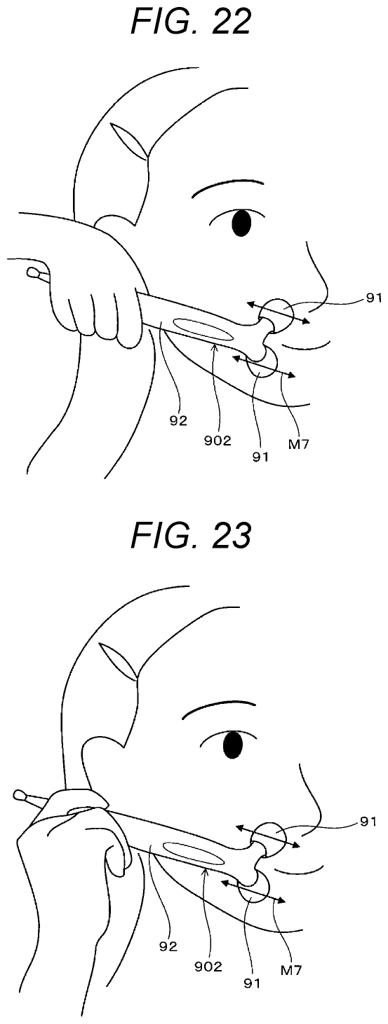

On the other hand, FIG. 22 illustrates a conventional cosmetic device 902 having a rod-shaped handle 92 and two rotating bodies 91 provided at the tip of the handle 92. In such a cosmetic device 902, when the two rotating bodies 91 are pressed against the skin, the longitudinal direction of the handle 92 is substantially orthogonal to the direction in which the two rotating bodies 91 are disposed. Therefore, in a case of performing a massage for picking up the skin using this type of cosmetic device, it is necessary to move the entire arm to reciprocate the cosmetic device 902 in the longitudinal direction of the handle 92 as illustrated with arrow M7. Therefore, the load on the arm is likely to be higher than that of the massager 1 of the first embodiment.

Further, in the conventional cosmetic device 902, as illustrated in FIG. 23, for example, there is a method for holding the handle 92 as by holding a pen, and reciprocating the cosmetic device 902 by shaking the forearm and the wrist. However, with this method, it is difficult to adjust the strength of the treatment on the skin. For example, the force with which the rotating body 91 presses the skin is likely to be weaker than when the main body 2 is gripped from the opposite side of the rotating body 3 as in the massager 1 of the first embodiment. For this reason, the power of picking up the skin may be weakened.

As described above, according to the massager 1 of the first embodiment, the strength of the treatment can be easily adjusted as compared with the conventional cosmetic device 902. Further, the load on the arm in a case of performing the massage can be further reduced, and the usability can be improved.

In the massager 1 of the first embodiment, a direction parallel to a straight line L3 connecting the second vertex 311b and the rear end point 241 is referred to as a longitudinal direction X as illustrated in FIG. 4. Further, a direction orthogonal to the straight line L3 in plan view viewed from the thickness direction of the main body 2 is referred to as a lateral direction Y. In this case, the external dimension DX of the main body 2 in the longitudinal direction X is longer than the external dimension DY of the main body 2 in the lateral direction Y.

Therefore, for example, as illustrated in FIGS. 14 and 16, the user grips the main body 2 from the opposite side of the rotating body 3, that is, from the left side in the width direction, and presses the rotating body 3 against the skin, thereby the pressing force to the skin can be appropriately increased. Further, by making the shape of the main body 2 long in the longitudinal direction as described above, the distance from the hand to the rotating body 3 can be reduced as compared with the case where the main body is long in the lateral direction.

Therefore, according to the massager 1 of the first embodiment, in a case of performing a massage for rolling the rotating body 3 to the skin or a massage for picking up the skin, the massager 1 becomes easy to move as intended while appropriately pressing the skin. As a result, the usability of the massager 1 can be further improved.

Further, as illustrated in FIG. 4, the main body 2 includes two regions A1 and A2 defined by a center line L4 in plan view viewed from the thickness direction. The center line L4 is a line connecting the centroids of the main body 2 in a cross section perpendicular to the straight line L3 connecting the second vertex 311b and the rear end point 241. In other words, the center line L4 is a line extending through the centroid of the main body 2 in a cross section perpendicular to the straight line L3. The two rotating bodies 3 are disposed outside the same area A1 of the two regions A1 and A2.

Therefore, for example, as illustrated in FIGS. 14 and 16, in a case of performing a massage for rolling the rotating body 3 on the skin or a massage for picking up the skin, the user grasps the main body 2 from the opposite side of the rotating body 3 and presses the rotating body 3 against the skin, so that the pressing force on the skin can be set moderately. In addition, by disposing the rotating body 3 at the specific position, the main body 2 can be gripped in a natural posture with the wrist straight, and the rotating body 3 can be pressed against the skin. As a result, the usability of the massager 1 can be further improved.

The main body 2 has a tapered shape in which the width in plan view viewed from the thickness direction becomes narrower from the second vertex 311b side toward the rear end point 241. Therefore, the shape of the main body 2 can be a shape that is more easily held by the user. As a result, the usability of the massager 1 can be further improved.

As illustrated in FIG. 2, the massager 1 includes two rotating bodies 3 disposed outside the main body 2 at intervals. The rotation axes 31 of the rotating bodies 3 extend so that the closer to the rotating body 3, the greater the distance between them. Therefore, as illustrated in FIGS. 16 and 18, for example, by moving the massager 1 back and forth on the skin while pressing the two rotating bodies 3 against the skin, massage can be performed so that the skin is picked up by the rotating bodies 3. And, by using the massager 1, the user can select a desired massage from among a scraping, a massage in which the rotating body 3 moves on the skin, a massage in which the scraping and the movement of the rotating body 3 performed simultaneously, and a massage in which the skin is picked up.

As illustrated in FIG. 2, the massager 1 includes a first electrode 41 disposed on the peripheral edge 21 of the main body 2, the second electrode 42 disposed on the surface of the rotating body 3, and the third electrode 43 disposed at a position separated from the first electrode 41 in the main body 2. In a state where at least one of the first electrode 41 and the second electrode 42 and the third electrode 43 are in contact with the skin, the massager 1 can form a current path including the electrode in contact with the skin.

Therefore, the main body 2 is grasped by hand so as to in contact with the third electrode 43, and at least one of the first electrode 41 and the second electrode 42 is pressed against the skin to perform a massage such as a scraping, so that a weak current can flow to the current path. In addition to the massage such as a scraping, the effect of the massage and the effect of the weak current act synergistically by applying the aforementioned weak current to the part being massaged, so that the cosmetic effect on the skin can be more improved.

### Second Embodiment

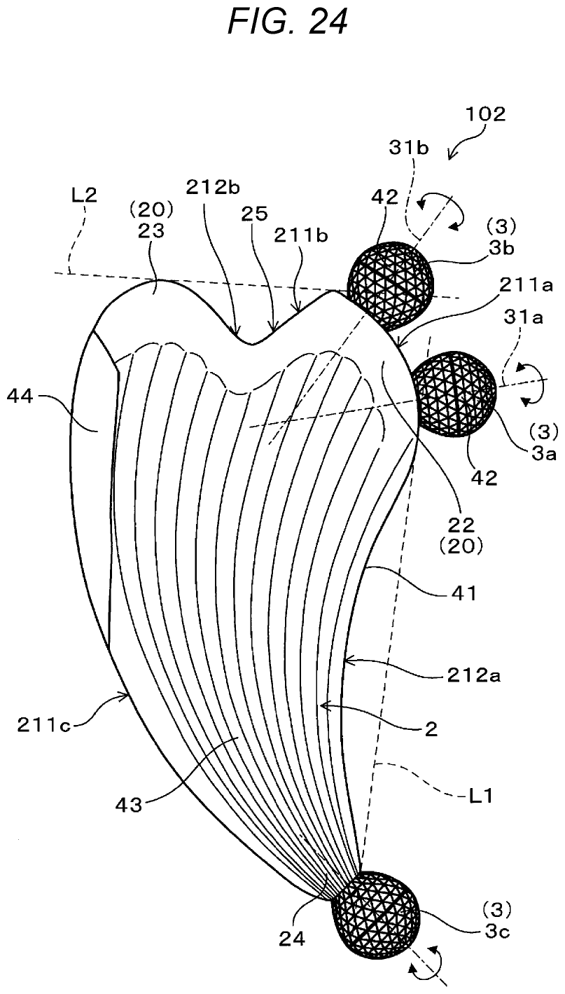

FIG. 24 illustrates a massager 102 according to a second embodiment. The massager 102 includes the main body 2 and the rotating bodies 3 (3a, 3c) provided at both ends of the first concave portion 212a of the main body 2. Among the reference numerals used in the second and subsequent embodiments, the same reference numerals as those used in the above-described embodiments denote the same components as those in the above-described embodiments unless otherwise specified.

As illustrated in FIG. 24, the massager 102 of the second embodiment includes the main body 2 that has a heart shape in plan view viewed from the thickness direction. A first rotating body 3a and a second rotating body 3b are disposed at an interval from each other on the first convex portion 211a of the peripheral edge 21 of the main body 2. Further, a third rotating body 3c is provided on the tail portion 24 which forms a part of the third convex portion 211c. The third rotating body 3c is disposed so as to protrude outward from the peripheral edge 21 of the main body 2. More specifically, the third rotating body 3c is disposed so as to protrude outward from the straight line L1 that is in contact with both the tail portion 24 as a part of the third convex portion 211c and the first convex portion 211a. Although not illustrated in the drawing, the rotation axis 31c of the third rotating body 3c extends in a direction orthogonal to the thickness direction of the main body 2. Other configurations are the same as in the first embodiment.

In the massager 102 of the second embodiment, the first rotating body 3a and the third rotating body 3c are provided at both ends of the first concave portion 212a. Therefore, for example, by simultaneously pressing the first concave portion 212a and the third rotating body 3c against the skin to reciprocate the massager 102, the massage for rolling the rotating body 3 can be performed simultaneously as the scraping. In addition, the massager 102 of the second embodiment can exhibit the same operational effects as the massager 1 of the first embodiment.

### Third Embodiment

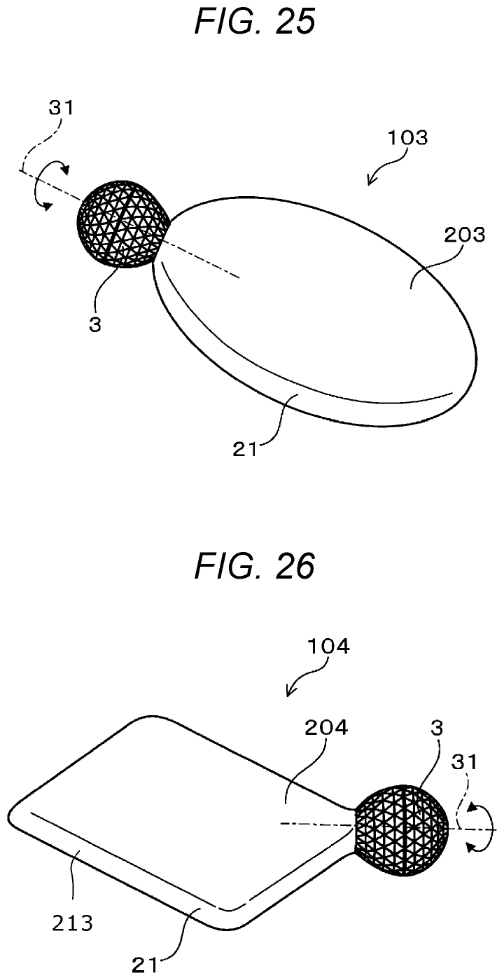

FIGS. 25 and 26 illustrate massagers 103 and 104 according to a third embodiment having a main body having a relatively simple shape. The main body of the massager may have a relatively complicated shape as in the first embodiment, or may have a relatively simple shape such as a disk shape or a square shape. For example, when the main body 203 has a disk shape as in the massager 103 illustrated in FIG. 25, the rotating body 3 can be provided at an arbitrary position on the peripheral edge 21 of the main body 203. Further, for example, when the main body 204 has a rectangular shape as in the massager 104 illustrated in FIG. 26, the rotating body 3 may be provided at a corner of the main body 204, or although not illustrated in the drawing, the rotating body 3 may be provided on the linear portion 213 of the 203.

Regardless of the shape of the main bodies 2, 203, and 204, the scraping and the massage for rolling the rotating body 3 can be performed simultaneously as long as the rotating body 3 is disposed so that the rotating body 3 and the peripheral edge 21 of the main bodies 2, 203, 204 can be pressed against the skin simultaneously.

### Fourth Embodiment

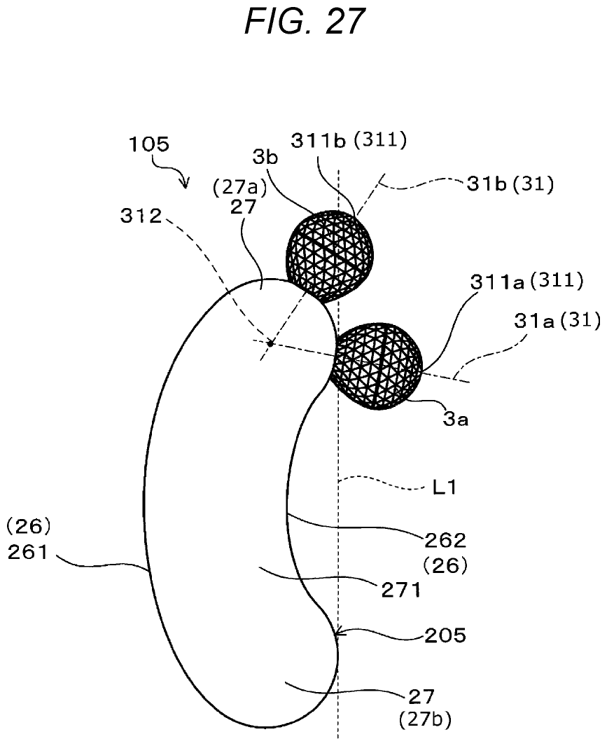

FIGS. 27 to 29 illustrate the massager 105 of a fourth embodiment. The massager 105 includes a plate-shaped main body 205 having an arc shape in plan view viewed from the thickness direction. The main body 205 includes both ends 27 (27a, 27b) and an intermediate portion 271 connecting the ends 27 together. Each end 27 has a semicircular shape in plan view viewed from the thickness direction. The intermediate portion 271 has a constant width over the entire length. Further, the peripheral edge 26 of the main body 205 includes a convex portion 261 and a concave portion 262 connected to the convex portion 261. The contour of the convex portion 261 in plan view viewed from the thickness direction of the main body 205 is convexly curved. The contour of the concave portion 262 in plan view is concavely curved.

Outside the one end 27a of the main body 205, two rotating bodies 3 are disposed so as to project outward from the peripheral edge 26 of the main body 205. The rotation axes 31 (31a, 31b) of the two rotating bodies 3 (3a, 3b) extend so as to increase the distance between them from the main body 205 toward the rotating body 3, and cross each other at the intersection 312.

As illustrated in FIG. 27, the main body 205 includes the concave portion 262 and the convex portions 261 connected to both ends of the concave portion 262. In the convex portion 261, a straight line that is in contact with both the portion closer to the rotating body 3 than the concave portion 262 and the portion on the opposite side to the rotating body 3 is referred to as the straight line L1. The first rotating body 3a near the concave portion 262 of the two rotating bodies 3 is disposed so as to protrude outward from the straight line L1.

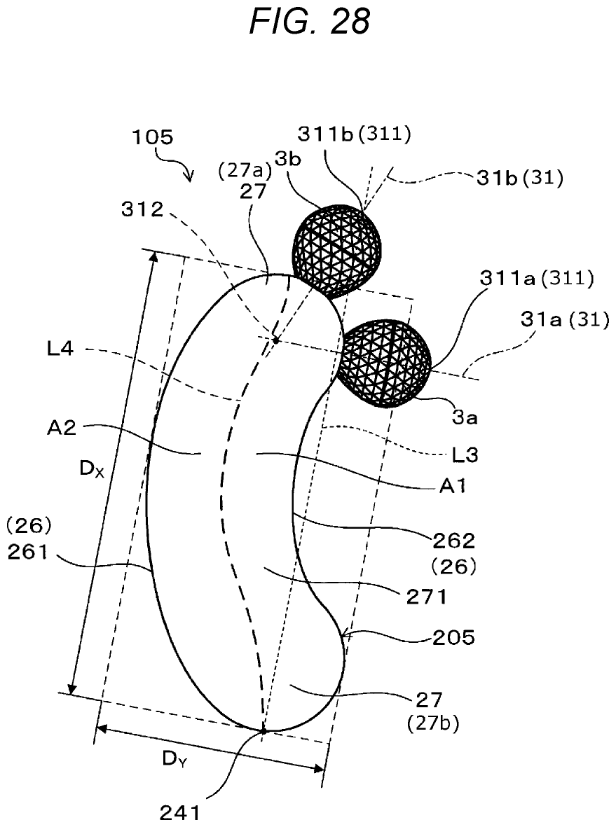

Further, as illustrated in FIG. 28, the two rotating bodies 3 are disposed side by side along the contour of the main body 205 in plan view viewed from the thickness direction. That is, the massager 105 of the fourth embodiment includes the two 0094] 311 (311a, 311b), the intersection 312, and the rear end point 241. One vertex (311a) of the two vertexes 311 is a point at which the outer surface of one rotating body (3a) of the two rotating bodies 3 intersects with the rotation axis (31a) of the rotating body (3a). The other vertex (311b) of the two vertexes 311 is a point at which the outer surface of the other rotating body (3b) of the two rotating bodies 3 intersects with the rotation axis (31b) of the rotating body (3b). The intersection 312 is an intersection between the rotation axes 31 of the two rotating bodies 3. The rear end point 241 is a point on the outer surface of the main body 2 where the distance from the intersection 312 is the largest.

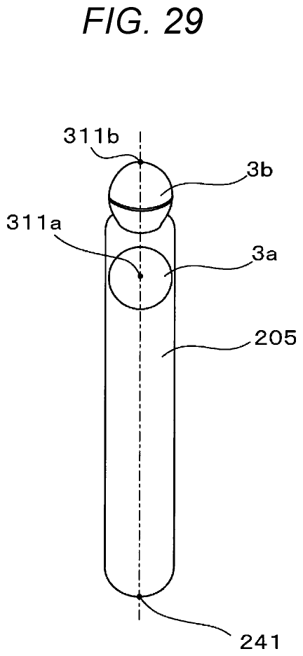

In the two vertexes 311 (311a, 311b), the vertex whose distance from the rear end point 241 is short (that is, the vertex of the first rotating body 3a) is referred to as the first vertex 311a, and the vertex whose distance from the rear end point 241 is long (that is, the vertex of the second rotating body 3b) is referred to as the second vertex 311b. As illustrated in FIG. 29, the first vertex 311a, the second vertex 311b, and the rear end point 241 are aligned in a straight line, and the first vertex 311a is located between the second vertex 311b and the rear end point 241 when viewed the massager 1 such that the distance from the second vertex 311b to the rear end point 241 is maximized. Although the surface shape of the rotating body 3 is simplified in FIG. 29 for convenience, the surface of the rotating body 3 is actually configured by a large number of triangular planes as illustrated in FIG. 28.

Further, as illustrated in FIG. 28, when a straight line connecting the second vertex 311b and the rear end point 241 is referred to as the straight line L3, a direction parallel to the straight line L3 is referred to as the longitudinal direction X, and a direction orthogonal to the straight line L3 in plan view viewed from the thickness direction of the main body 2 is referred to as the lateral direction Y, the external dimension DX of the main body 205 in the longitudinal direction X is longer than the external dimension DY of the main body 205 in the lateral direction Y.

The main body 205 includes two regions A1 and A2 defined by the center line L4 in plan view when viewed from the thickness direction of the main body 205. The two rotating bodies 3 are disposed outside the same area A1 of these two regions A1 and A2. Other configurations are the same as in the first embodiment.

The massager 105 of the fourth embodiment can be used as the massager 1 of the first embodiment except for a method for sandwiching a portion that protrudes outward from the periphery of the chin or the like between the first top portion 22 and the second top portion 23.

### Fifth Embodiment

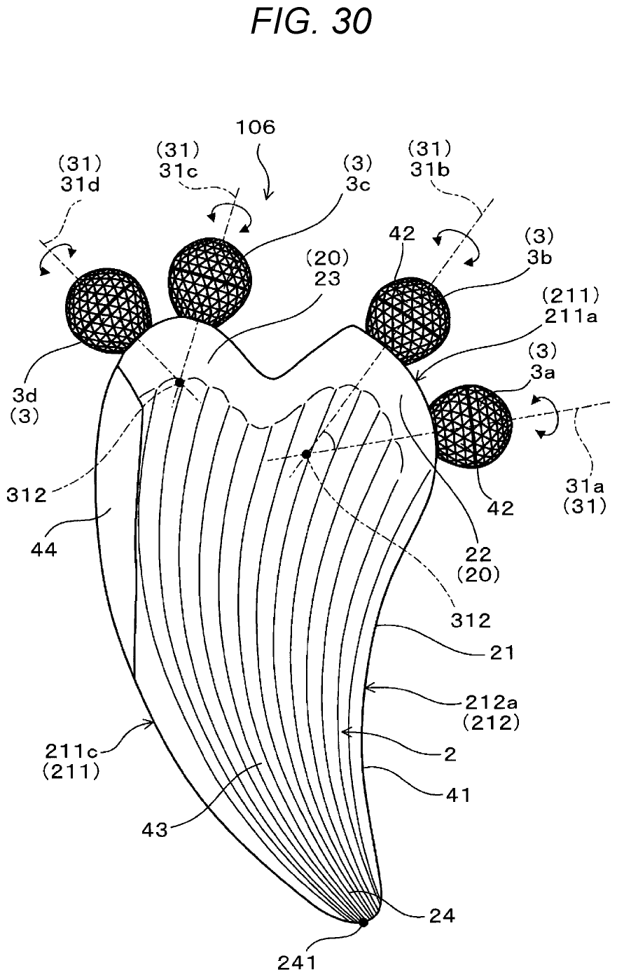

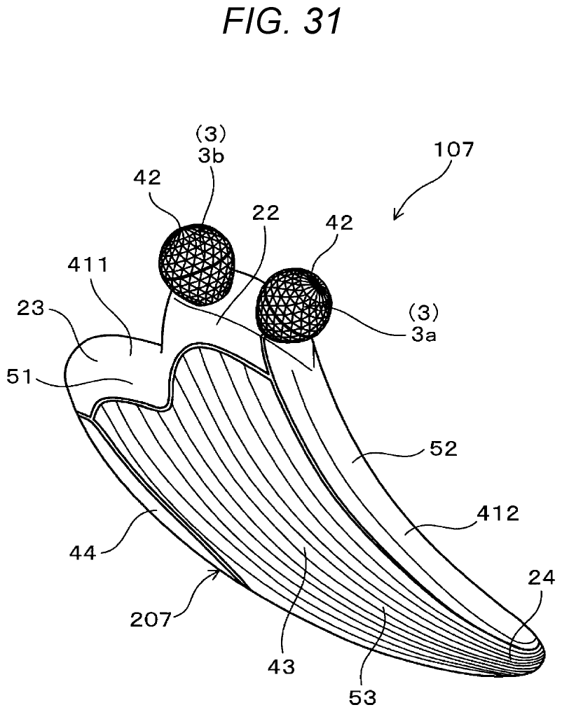

FIG. 30 illustrates a massager 106 according to a fifth embodiment in which the first top portion 22 and the second top portion 23 each include the two rotating bodies 3. That is, massager 106 includes the two rotating bodies 3 (3c, 3d) at the second top portion 23 in addition to the first top portion 22. The rotation axes 31 (31c, 31d) of the two rotating bodies 3 (3c, 3d) extend so as to increase the distance between them from the main body 2 toward the rotating body 3, and cross each other at the intersection 312. Other configurations are the same as in the first embodiment.

The massager 106 of the fifth embodiment, like the massager 1 of the first embodiment, can perform a massage for picking up the skin using the two rotating bodies 3a and 3b provided on the first top portion 22. Further, a massage for picking up the skin can be performed using the two rotating bodies 3c and 3d provided on the second top portion 23. Further, by using the massager 106, similarly to the massager 1 of the first embodiment, various massages such as a method for simultaneously performing the scraping and the massage with the rotating body 3, the scraping along, and the massage with the rotating body 3 alone can be performed.

### Sixth Embodiment

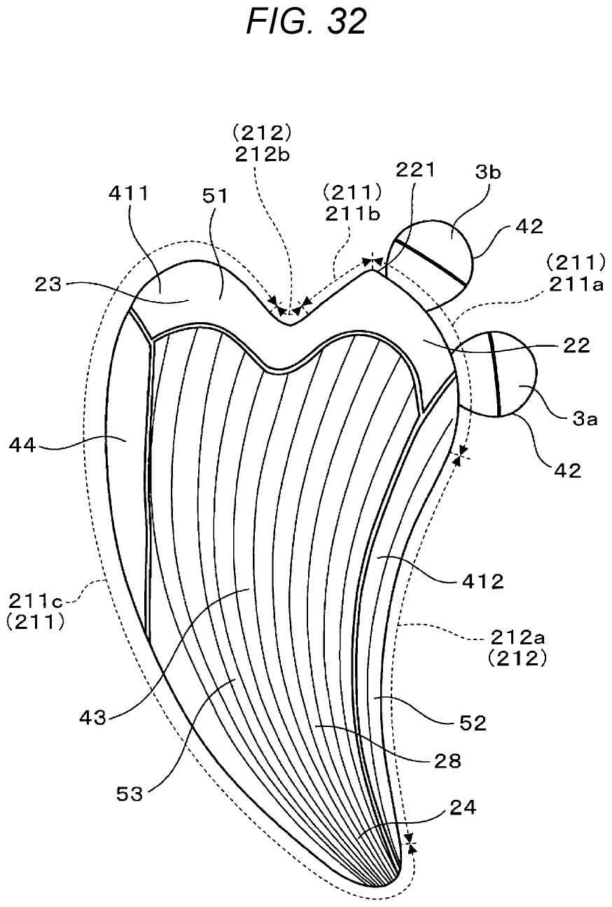

FIGS. 31 and 32 illustrate a massager 107 of a sixth embodiment in which first electrodes 411 and 412, the second electrode 42, and the third electrode 43 are provided on separate components. The massager 107 has a shape similar to that of the massager 1 of the first embodiment, that is, a heart shape in plan view viewed from the thickness direction.

The outer surface of a main body 207 includes three shells 51 to 53. The first shell 51 is disposed on the upper portion of the main body 2. As illustrated in FIG. 32, the first shell 51 includes an upper portion of the first convex portion 211a, the second convex portion 211b, the second concave portion 212b, and an upper portion of the third convex portion 211c. Further, a first electrode 411 is disposed on the surface of the first shell 51.

The second shell 52 is disposed on the right portion of the main body 2. The second shell 52 includes a lower portion in the first convex portion 211a and the first concave portion 212a. Further, a first electrode 412 is disposed on the surface of the second shell 52.

Portions other than the first shell 51, the second shell 52, and the solar cell module 44 on the outer surface of the main body 2 are configured by the third shell 53. That is, the two main surface portions 28 disposed on both surfaces in the thickness direction of the main body 2 and the portion of the third convex portion 211c below the solar cell module 44 are formed by the third shell 53. The third electrode 43 is disposed on the surface of the third shell 53.

The first electrode 411 of the first shell 51, the first electrode 412 of the second shell, and the second electrode 42 provided on the surface of the rotating body 3 are electrically connected to each other inside the main body 207. Furthermore, these electrodes 411, 412, and 42 are electrically connected to the solar cell module 44. The third electrode 43 provided on the surface of the third shell 53 is electrically connected to the solar cell module 44. Other configurations are the same as in the first embodiment.

The massager 107 of the sixth embodiment can achieve the same operational effects as those of the first embodiment.

The embodiment of the massager according to the invention is not limited to the first to sixth embodiments described above, and the configuration can be appropriately changed without impairing its purpose.

### REFERENCE SIGNS LIST

1, 102, 103, 104, 105, 106, 107 massager
2, 203, 204, 205, 207 main body
21, 26 peripheral edge
3 rotating body
31 rotation axis

1. A massager, comprising:
a main body that has a plate shape, which can apply a massage by sliding a peripheral edge of the main body on the skin; and
at least one rotating body that is rotatable and disposed to protrude outward from the peripheral edge of the main body,
wherein a rotation axis of the rotating body extends outward through the peripheral edge of the main body.

2. The massager according to claim 1,
wherein the main body includes at least one bulging portion that bulges outward in plan view viewed from a thickness direction of the main body, and
wherein the rotating body is disposed to protrude outward from the bulging portion.

3. The massager according to claim 2,
wherein the at least one bulging portion includes two bulging portions disposed side by side, and
wherein the main body includes the two bulging portions and a depressed portion that is formed between the two bulging portions and is depressed more inward than the two bulging portions in plan view viewed from the thickness direction.

4. The massager according to claim 1,
wherein the rotating body has a spherical shape.

5. The massager according to claim 1,
wherein the at least one rotating body includes two rotating bodies that are disposed outside the main body with a space between the two rotating bodies, and
wherein rotation axes of the two rotating bodies extend so that a distance between the two rotating bodies increases from the main body toward the two rotating bodies.

6. The massager according to claim 5, wherein the two rotating bodies are disposed side by side along a contour of the main body in plan view viewed from a thickness direction of the main body.

7. The massager according to claim 6, comprising:
two vertexes, one of the two vertexes being a point where an outer surface of one of the two rotating bodies and the rotation axis of the rotating body intersect, and the other of the two vertexes being a point where an outer surface of the other of the two rotating bodies intersects with the rotation axis of the rotating body;
an intersection between the rotation axes of the two rotating bodies; and
a rear end point that is a point at which a distance from the intersection on an outer surface of the main body is the largest,
wherein, when a vertex having a shorter distance from the rear end point of the two vertexes is referred to as a first vertex, a vertex of the two vertexes having a longer distance from the rear end point is referred to as a second vertex, a direction parallel to a straight line connecting the second vertex and the rear end point is referred to as a longitudinal direction, and a direction orthogonal to the straight line in plan view viewed from the thickness direction of the main body is referred to as a lateral direction, an external dimension of the main body in the longitudinal direction is longer than an external dimension of the main body in the lateral direction.

8. The massager according to claim 6, comprising:
two vertexes, one of the two vertexes being a point where an outer surface of one of the two rotating bodies and the rotation axis of the rotating body intersect, and the other of the two vertexes being a point where an outer surface of the other of the two rotating bodies intersects with the rotation axis of the rotating body;
an intersection between the rotation axes of the two rotating bodies; and
a rear end point that is a point at which a distance from the intersection on an outer surface of the main body is the largest,
wherein, when a vertex having a shorter distance from the rear end point of the two vertexes is referred to as a first vertex, and a vertex of the two vertexes having a longer distance from the rear end point is referred to as a second vertex, the main body includes two regions defined by a center line in plan view viewed from the thickness direction, the center line is a line connecting centroids of the main body in a cross section perpendicular to a straight line connecting the second vertex and the rear end point, and
wherein the two rotating bodies are disposed outside a same region of the two regions.

9. The massager according to claim 6, comprising:
two vertexes, one of the two vertexes being a point where an outer surface of one of the two rotating bodies and the rotation axis of the rotating body intersect, and the other of the two vertexes being a point where an outer surface of the other of the two rotating bodies intersects with the rotation axis of the rotating body;
an intersection between the rotation axes of the two rotating bodies; and
a rear end point that is a point at which a distance from the intersection on an outer surface of the main body is the largest,
wherein, when a vertex having a shorter distance from the rear end point of the two vertexes is referred to as a first vertex, and a vertex of the two vertexes having a longer distance from the rear end point is referred to as a second vertex, the main body includes has a tapered shape in which a width in plan view viewed from the thickness direction becomes narrow from the second vertex side toward the rear end point.

10. A massaging method comprising:
sliding the peripheral edge of the massager of claim 1 across the skin of a subject being massaged when the at least one rotating body is rotating.
